(19) United States
(12) Patent Application Publication
(10) Pub. No.: US 2019/0324047 A1
(43) Pub. Date: Oct. 24, 2019

(54) JUST IN TIME AVAILABILITY OF ANALYTICAL TEST RESULTS

(71) Applicant: Roche Diagnostics Operations, Inc., Indianapolis, IN (US)

(72) Inventors: Louis Depraetere, Erpent (BE); Alexander Lefevre, Vorselaar (BE); Janne Henrike Paerssinen, Braine-l'Alleud (BE); Annick Tourny, Vosselare (BE); Marc Vanderkeel, Heverlee (BE); Katleen Vandeweyer, Uccle (BE); Werner Vercammen, Putte (BE)

(73) Assignee: Roche Diagnostics Operations, Inc., Indianapolis, IN (US)

(21) Appl. No.: 16/380,507

(22) Filed: Apr. 10, 2019

(30) Foreign Application Priority Data
Apr. 20, 2018 (EP) 18168360.8

(51) Int. Cl. G01N 35/00; G16H 80/00

(52) U.S. Cl. CPC G01N 35/0092; G01N 35/00871; G01N 2035/00891; G16H 80/00; G01N 2035/00881; G01N 35/00732

(57) ABSTRACT

A computer implemented method for operating an analytical laboratory for processing biological samples is presented. The processing priority of test orders related to the biological samples is determined based on an objectively determined urgency of the analytical test results and on staff workload at a forecast time of availability of the analytical test result(s).

### CROSS-REFERENCE TO RELATED APPLICATIONS

This application claims priority to EP 18168360.8, filed Apr. 20, 2018, which is hereby incorporated by reference.

### BACKGROUND

The present disclosure generally relates to a computer implemented method for operating an analytical laboratory for processing biological samples.

In vitro analytical diagnostic testing has a major effect on clinical decisions, providing physicians with pivotal information. Statistics reveal that a very high proportion of all physician decisions are dependent on in vitro diagnostic testing.

In analytical laboratories, in particular clinical laboratories, a multitude of analyses on samples are executed by an analytical system in order to determine the physiological state of a patient. The kind of analytical test to be executed on a biological sample is typically specified as a test order which is typically registered in a laboratory information system as a record and sent to the analytical laboratory.

According to established laboratory procedures, when a biological sample is received, it is first identified—for example, by an identifier label and corresponding label reader such as a barcode label and barcode reader. Once the biological sample is identified, an order list is retrieved from a database comprising a plurality of test orders, each test order defining one or more processing steps to be carried out on the biological sample by one or more of the laboratory instruments. These processing steps may be pre-analytical processing steps (such as aliquoting, sample preparation), analytical processing steps (such as an assay to determine the presence and/or concentration of an analyte in the biological sample) or post-analytical processing steps (such as archiving of the biological sample). Before the biological sample can be processed by the various laboratory instruments of the analytical laboratory, a processing workflow is determined. The processing workflow is defined as a sequence in which the test orders are to be processed and/or a timing of processing of the test orders.

According to current practice, samples, i.e., test orders which need to be processed and analyzed very urgently—as the analysis result may be of life-critical importance for a patient—are marked as urgent STAT samples. STAT laboratory tests and services are those that are needed immediately in order to manage medical emergencies. According to current laboratory practice, STAT test requests are given the highest priority by the analytical laboratory for processing, analysis and reporting.

Health care institutions (such as hospitals, clinics, medical practices, home-based offices of medical practitioners and the like) have to manage a number of limited medical practitioners (e.g. physicians, nurses, etc.) to be able to treat an ever increasing number of patients efficiently and effectively. Uncertain time of availability of results of analytical test leads to lack of efficiency as physicians have to wait on results of analytical test(s) to become available to be able to determine a diagnosis/treatment or need for further analytical tests i.e., medical practitioners need to check multiple times whether results for an analytical test have become available for the most urgent patients, or go through a long list of already processed lab results and related alerts—that they might not have had time to review yet.

Furthermore, not only uncertain time of availability of results of analytical test(s), but in addition, late/delayed availability of results of analytical tests are a cause of lack of efficiency of such health care institutions. This uncertainty regarding time of arrival of results in an increased risk of a negative outcome for the patient. In order to ensure timely availability of results of analytical test(s), according to currently known practice, physicians would order analytical test(s) on a sample of a patient as urgent STAT whenever they decide (based on certain rules or personal judgement) that the particular analytical test or patient sample should be prioritized. Factors influencing the physician's decision to classify a sample or test order (for analytical test) as urgent include—but not limited to—patient severity (or indication thereof) and/or patient history. However, if less urgent tests are also ordered STAT or if too many urgent test orders are issued in a timeframe, a backlog of the analytical laboratory may develop if the number of urgent test orders exceeds the throughput of the laboratory and hence each sample will be delayed, including urgent ones.

Therefore, there is a need for an analytical laboratory and a method of operating the analytical laboratory in such a way as to allow for efficient use of resources of health care institutions to improve patient care and/or reduce costs.

### SUMMARY

According to the present disclosure, a computer implemented method for operating an analytical laboratory is presented. The analytical laboratory can comprise a plurality of laboratory instruments configured to obtain a measurement value indicative of a characteristic of the biological sample. The method can comprise receiving and identifying a plurality of biological samples and retrieving one or more test orders corresponding to each biological sample. Each test order can define at least one analytical step to be carried out on the biological sample by at least one of the plurality of the laboratory instruments. Each test order can comprise a processing priority. The method can also comprise determining a processing workflow for processing each biological sample based on corresponding test order(s) and corresponding processing priority. The processing workflow can be determined such that test order(s) with higher processing priority are completed before test order(s) with lower processing priority. The method can also comprise determining a forecast availability of analytical result(s) corresponding to each test order, determining a staff workload and a workload threshold at the time of forecast availability corresponding to each test order, decreasing the processing priority corresponding to the respective test order if the staff workload is greater than a workload threshold, increasing the processing priority corresponding to the respective test order if the staff workload is lower than the workload threshold, and controlling the plurality of laboratory instruments of the analytical laboratory to process each biological sample according to the corresponding processing workflow and reporting test results obtained by the at least one analytical step on the biological samples performed by the at least one of the plurality of the laboratory instruments if the staff workload matches the workload threshold.

Accordingly, it is a feature of the embodiments of the present disclosure to provide an analytical laboratory and a method of operating the analytical laboratory in such a way as to allow for efficient use of resources of health care institutions to improve patient care and/or reduce costs. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a flowchart for a method for operating an analytical laboratory according to an embodiment of the present disclosure.

FIG. 2 illustrates a flowchart for a method for operating an analytical laboratory wherein the processing priority of a test order is set initially based on an objectively determined urgency level according to an embodiment of the present disclosure.

FIG. 3 illustrates a flowchart for a method for operating an analytical laboratory wherein machine learning is employed to revise parameters for determining forecast availability of analytical results if the actual availability of analytical result(s) deviate from the forecast availability according to an embodiment of the present disclosure.

FIGS. 4A-B illustrate a flowchart for a method for operating an analytical laboratory, wherein the biological sample(s) is transferred to a further analytical laboratory if the forecast availability of analytical result(s) corresponding to the test order(s) for that biological sample is beyond a specified availability deadline according to an embodiment of the present disclosure.

FIG. 5 illustrates a flowchart for a method for operating an analytical laboratory, wherein a phlebotomist is alerted to collect a new patient sample if at least one analytical step of the test order(s) cannot be performed on a biological sample due to sample quality/quantity according to an embodiment of the present disclosure.

FIG. 6 illustrates a flowchart for a method for operating an analytical laboratory, wherein the forecast availability(s) of analytical result(s) is reported when being determined and re-determined according to an embodiment of the present disclosure.

FIG. 7 illustrates a flowchart for a method for operating an analytical laboratory, wherein the processing priority level of all test orders is set to the highest level amongst the test orders corresponding to the same patient, same doctor and/or same hospital according to an embodiment of the present disclosure.

FIG. 8 illustrates a highly schematic block diagram of an analytical laboratory according to an embodiment of the present disclosure.

FIG. 9 illustrates a mockup of an example of a mobile device application reporting the forecast availability of analytical results in the form of progress bars according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

Certain terms will be used in this patent disclosure, the formulation of which should not be interpreted to be limited by the specific term chosen, but as to relate to the general concept behind the specific term.

The term ‘laboratory instrument’ as used herein can encompass any apparatus or apparatus component operable to execute one or more processing steps/workflow steps on one or more biological samples. The expression ‘processing steps’ thereby can refer to physically executed processing steps such as centrifugation, aliquotation, sample analysis and the like. The term ‘laboratory instrument’ can cover pre-analytical instruments, post-analytical instruments and also analytical instruments.

The term ‘pre-analytical instrument’ as used herein can comprise one or more lab-devices for executing one or more pre-analytical processing steps on one or more biological samples, thereby preparing the samples for one or more succeeding analytical tests. A pre-analytical processing step can be, for example, a centrifugation step, a capping-, decapping- or recapping step, an aliquotation step, a step of adding buffers to a sample and the like. The expression ‘analytical system’ as used herein can encompass any monolithic or multi-modular laboratory device comprising one or more lab-devices or operative units which can be operable to execute an analytical test on one or more biological samples.

The term ‘post-analytical instrument’ as used herein can encompass any laboratory instrument being operable to automatically process and/or store one or more biological samples. Post-analytical processing steps may comprise a recapping step, a step for unloading a sample from an analytical system or a step for transporting the sample to a storage unit or to a unit for collecting biological waste.

The term ‘analyzer’/‘analytical instrument’ as used herein can encompass any apparatus or apparatus component configured to obtain a measurement value. An analyzer can be operable to determine via various chemical, biological, physical, optical or other technical procedures a parameter value of the sample or a component thereof. An analyzer may be operable to measure the parameter of the sample or of at least one analyte and return the obtained measurement value. The list of possible analysis results returned by the analyzer can comprise, without limitation, concentrations of the analyte in the sample, a digital (yes or no) result indicating the existence of the analyte in the sample (corresponding to a concentration above the detection level), optical parameters, DNA or RNA sequences, data obtained from mass spectrometry of proteins or metabolites and physical or chemical parameters of various types. An analytical instrument may comprise units assisting with the pipetting, dosing, and mixing of samples and/or reagents. The analyzer may comprise a reagent holding unit for holding reagents to perform the assays. Reagents may be arranged for example in the form of containers or cassettes containing individual reagents or group of reagents, placed in appropriate receptacles or positions within a storage compartment or conveyor. It may comprise a consumable feeding unit. The analyzer may comprise a process and detection system whose workflow can be optimized for certain types of analysis. Examples of such analyzer are clinical chemistry analyzers, coagulation chemistry analyzers, immunochemistry analyzers, urine analyzers, nucleic acid analyzers, tissue analyzers (including morphological stainers and histochemical stainers) used to detect the result of chemical or biological reactions or to monitor the progress of chemical or biological reactions.

The term ‘analytical laboratory’ as used herein can encompass any system for the use in a laboratory comprising a plurality of laboratory instruments operatively connected to a control unit.

The term ‘control unit’ as used herein can encompass any physical or virtual processing device configurable to control an analytical laboratory comprising a plurality of laboratory instruments in a way that a workflow and workflow step(s) can be conducted by the analytical laboratory. The control unit may, for example, instruct the analytical laboratory (or a specific instrument thereof) to conduct pre-analytical, post analytical and analytical workflow(s)/workflow step(s). The control unit may receive information from a data management unit regarding which steps need to be performed with a certain sample. In some embodiments, the control unit may be integral with a data management unit, may be comprised by a server computer and/or be part of one instrument or even distributed across multiple instruments of the analytical laboratory. The control unit may, for instance, be embodied as a programmable logic controller running a computer-readable program provided with instructions to perform operations.

The term ‘communication network’ as used herein can encompass any type of wireless network, such as a WIFI, GSM, UMTS or a cable based network, such as Ethernet or the like. In particular, the communication network can implement the Internet protocol IP. For example, the communication network can comprise a combination of cable-based and wireless networks. In embodiments wherein units of the system are comprised within one laboratory instrument, the communication network can comprise communication channels within an instrument.

The term ‘user interface’ as used herein can encompass any suitable piece of software and/or hardware for interactions between an operator and a machine, including but not limited to a graphical user interface for receiving as input a command from an operator and also to provide feedback and convey information thereto. Also, a system device may expose several user interfaces to serve different kinds of users/operators.

The term ‘workflow’/‘processing workflow’ as used herein can refer to a collection of workflow steps/processing steps. According to particular embodiments, the workflow can define a sequence in which the processing steps are carried out.

The term ‘workflow step’ or ‘processing step’ as used herein can encompass any activity belonging to a workflow. The activity can be of an elementary or complex nature and can be typically performed at or by one or more instrument(s).

The terms ‘sample’, ‘patient sample’ and ‘biological sample’ can refer to material(s) that may potentially contain an analyte of interest. The patient sample can be derived from any biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cultured cells, or the like. The patient sample can be pretreated prior to use, such as preparing plasma from blood, diluting viscous fluids, lysis or the like. Methods of treatment can involve filtration, distillation, concentration, inactivation of interfering components, and the addition of reagents. A patient sample may be used directly as obtained from the source or used following a pretreatment to modify the character of the sample. In some embodiments, an initially solid or semi-solid biological material can be rendered liquid by dissolving or suspending it with a suitable liquid medium. In some embodiments, the sample can be suspected to contain a certain antigen or nucleic acid.

A ‘STAT sample’ can be a sample which needs to be processed and analyzed very urgently as the analysis result may be of life-critical importance for a patient. STAT laboratory tests and services can be those that are needed immediately in order to manage medical emergencies.

The term ‘sample tube’ can refer to any individual container for transporting, storing and/or processing a sample. In particular, the term without limitation can refer to a piece of laboratory glass- or plastic-ware optionally comprising a cap on its upper end.

The term ‘analyte’ as used herein can refer to a component of a sample to be analyzed, e.g., molecules of various sizes, ions, proteins, metabolites and the like. Information gathered on an analyte may be used to evaluate the impact of the administration of drugs on the organism or on particular tissues or to make a diagnosis. Thus, ‘analyte’ can be a general term for substances for which information about presence and/or concentration is intended. Examples of analytes are e.g., glucose, coagulation parameters, endogenic proteins (e.g., proteins released from the heart muscle), metabolites, nucleic acids and so on.

The term ‘analysis’ or ‘analytical test’ as used herein can encompass a laboratory procedure characterizing a parameter of a biological sample, e.g., light absorption, fluorescence, electrical potential or other physical or chemical characteristics of the reaction to provide the measurement data.

The term ‘test order’ as used herein can refer to any data object, computer loadable data structure, modulated data representing such data indicative of one or more laboratory processing steps to be executed on a particular biological sample. For example, a test order record may be a file or an entry in a database. According to embodiments disclosed herein, a test order can indicate a test order for an analytical test if, for example, the test order comprises or is stored in association with an identifier of an analytical test to be executed on a particular sample. Alternatively, or additionally, the test order may refer to pre- and/or post-analytical processing steps to be performed on the biological sample.

The terms ‘analytical result’, ‘test result’ or ‘result of analytical test’ as used herein can encompass any data that is descriptive of a result of a measurement of an analyte in a biological sample, namely qualitative and/or quantitative measurement of an analyte in a sample. In particular, the analytical data can comprise an identifier of the sample for which the analysis has been performed and data descriptive of a result of the analysis, such as measurement data. Furthermore, the terms ‘analytical result’, ‘test result’ or ‘result of analytical test’ can also encompass calculated results based on measurement of an analyte in a biological sample, as risk scores, or other values calculated using various clinical decision support algorithms.

It has been observed that there can be situations in patient care institutions when a medical practitioner (and patient) has to wait for result(s) of analytical test order(s) longer than expected, while at the same time results of other analytical test orders can be available well before a medical practitioner actually has time to consider them.

Results of analytical tests which are not available when a medical practitioner would need them (in order to diagnose and/or treat a patient, to decide on further analytical testing, and the like) can be hereinafter referred to as delayed results.

Results of analytical tests which are available before they are needed/considered by a medical practitioner can be hereinafter referred to as early results.

The main reason for a test order to lead to an early result is that medical practitioners of a health care institution order analytical tests as urgent if they judge (based on certain rules or personal judgement) that the particular analytical test or patient sample should be prioritized, but lack resources (most commonly the time of medical practitioners) to consider these when the results are available. It has been observed that multiple times more test orders are ordered as urgent compared to the number of actual emergency cases in a typical health care institution. In other words, early results are just sitting idle until the medical practitioner has time to consider them. Also, medical practitioners may order a laboratory test around the end of their work shift and hence often the results are available early as the medical practitioners are not working anymore by the time the test results become available.

It has been recognized that early results hold back the processing of other more urgent test orders. In other words, it has been recognized that occurrence of delayed results can be greatly reduced by the analytical laboratory re-evaluating the priority level (urgent STAT vs. normal; or less urgent than STAT) of the analytical test orders/samples as defined at ordering (e.g., by the medical practitioner). For such re-evaluation of priority level by the analytical laboratory, a forecast of expected time of availability of test results can be calculated for each analytical test order followed by an adjustment of the processing priority thereof based on the staff workload/backlog at the time of forecast availability. The term staff workload/backlog can refer to the sum of all actions and resources needed before the analytical results can be actually considered. The staff workload/backlog may represent the number of other patients a medical practitioner must handle before having time to treat/diagnose the patient in question. The staff workload/backlog may also represent the period of time until a medical practitioner starts his/her work shift.

Potentially life-critical test results can be reviewed by the medical practitioner, or proxy, immediately—hence the staff workload/backlog vs. the current test order can always be zero/empty. In other words, treatment of patients in life-critical situations—and thus also consideration of related analytical test orders—can always be handled first.

At the onset—when the analytical laboratory retrieves a test order, the priority level of an analytical test corresponds to the priority level can be set by the orderer, most likely, a medical practitioner. The processing priority of an analytical test order can be reduced if at the time of forecast expected availability of the test results the medical practitioner will already have a high staff workload/backlog. On the other hand, if the staff workload/backlog of the medical practitioner at the forecast expected availability of the test results is low (meaning the medical practitioner is most likely to need the results right away, otherwise valuable time is wasted), the processing priority of an analytical test order can be raised or maintained if already set to urgent. According to particular embodiments disclosed herein, the forecast of expected time of availability of test results can be recalculated for each analytical test order and the priority level of all test orders can be re-assessed in an iterative manner each time the processing priority of a test order is changed. In another words, the impact of each change of processing priority of a test order on the forecast of all analytical tests can be assessed.

As often laboratory personnel needs to process (e.g., validate) the test results before being available to the medical practitioner, to account for possible delays due to such processing by laboratory personnel, according to embodiments disclosed herein, in addition to the staff workload/backlog of medical practitioner, the staff workload/backlog of laboratory personnel can be taken into account in scheduling of the processing workflow of analytical test orders as well as in calculating the forecast availability of the test results.

All in all, the analytical laboratory, as well as the method of operating an analytical laboratory disclosed herein can enable significantly more efficient operation of health care institutions by accurate forecasting of availability of laboratory test result(s) in combination with staff workload/backlog based prioritization and scheduling of the processing workflow of analytical test orders.

Embodiments of the disclosed method/system can be particularly advantageous since the occurrence of delayed results can be significantly reduced without any additional investments such as increasing throughput of the analytical laboratory (e.g., by additional instruments, higher throughput instruments and the like). In other words, a traditional first come first served approach with some pre-defined prioritization of processing test orders for analytical tests can be substituted with a prioritization corresponding to the actual needs of the health care institution. Reduction of delayed results as well as increasing the forecasting accuracy of test result availability can lead to a significantly better quality of patient care (reduced length of stay of a patient in a health care institution due to timely and/or quicker delivery of life-critical test results—in particular in emergency care as well as financial benefits (better use of existing resources, reduced cost due to less time spent in a health care establishment by patients). In addition, embodiments disclosed herein can support health care institutions to better cope with peaks of patient influx (epidemics, bio-terrorism) in that timely availability of analytical results can ensure healthy patients can be released sooner, thereby freeing up resources.

Due to the uncertain time of availability of results of analytical test, medical practitioners often perform point of care analytical testing instead of issuing a test order to an analytical laboratory. However, point of care analytical testing costs significantly more than the same analytical test performed by an analytical laboratory. Furthermore, point of care testing can sometimes provide less accurate analytical results as compared to laboratory testing. By reducing the uncertainty of availability of analytical test results, embodiments herein disclosed can further contribute to the reduction of operational costs and better quality of patient care since medical practitioners are less motivated to replace laboratory testing with point of care testing.

In addition, reporting a forecast of expected time of availability of test results can support medical practitioners of health care institutions to much better plan their activities. Better planning of activities of medical practitioners can inherently increase cost effectiveness and also improve the quality of care as timely availability of results of analytical tests can have a significant impact on decisions influencing patient care. Timely availability of results of analytical tests can allow for speedier treatment and may reduce the risks of complications, hence improving patient care. On the other hand, timely availability of results of analytical tests can also contribute to reducing costs as patients with less severe conditions may leave the health care institution earlier, while patients with more severe conditions are diagnosed earlier which very often is critical in effectively treating severe conditions.

According to further embodiments disclosed herein, the processing priority level of a test order can be adjusted also based on a processing status of the test order to account for unexpected events, such as low sample quality, rejected test result in need for a rerun of the analytical test, hardware failures of an instrument, and the like.

According to further embodiments disclosed herein, the processing priority level of a test order can be adjusted also based on the type of test order to ensure that analytical tests which are known to be required for life-critical diagnoses can be performed with highest priority, e.g., analytical tests needed to diagnose a heart-attack, an imminent sepsis and the like.

According to embodiments disclosed herein, the forecasting of availability of laboratory test results can be based on parameters which can be revised based on feedback indicative of actual time of availability of results of past test orders.

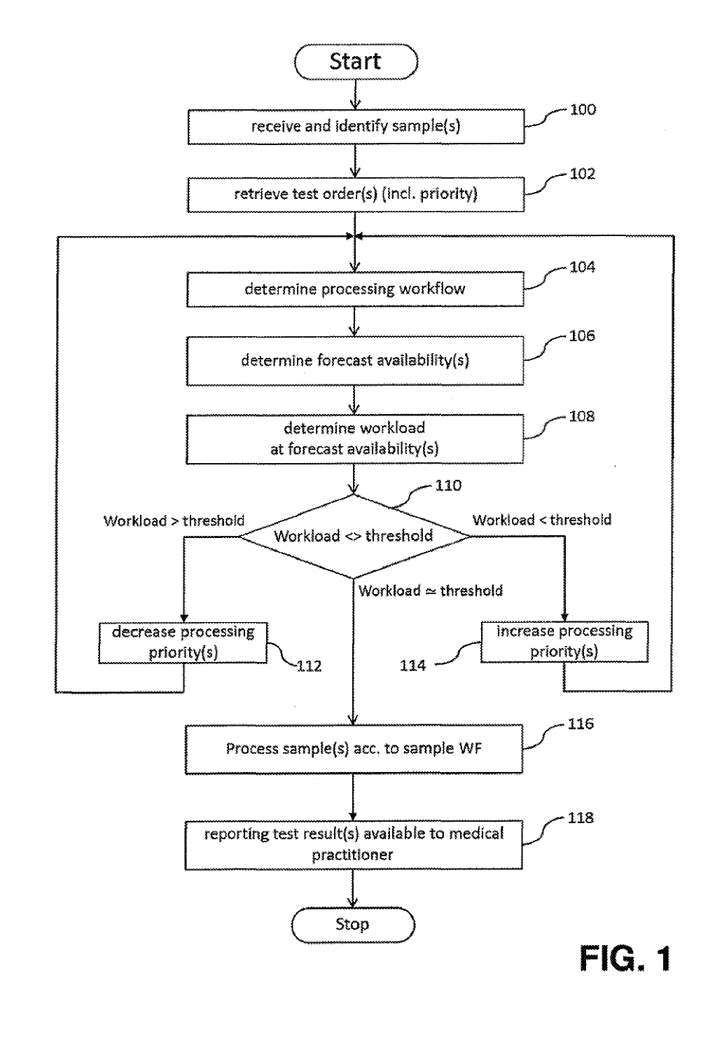

A first embodiment of the method for operating an analytical laboratory 1 can now be described as illustrated on the flowchart of FIG. 1.

In a step 100, one of the plurality of laboratory instruments 10 of the analytical laboratory 1 can receive and identify the biological sample. The biological sample can be identified such as, for example, based on an identification label attached to a sample tube holding the biological sample. The identification label attached may be a barcode, an RFID tag, or the like.

Once the biological sample is identified, in step 102, the control unit 20 can retrieve one or more test orders corresponding to each biological sample, each test order defining at least one analytical step to be carried out on the biological sample by at least one of the plurality of the laboratory instruments 10. According to some embodiments of the disclosed method/system, several processing steps may be carried out at the same test order, such as several aliquots to be prepared from the biological sample or several analytical tests to be carried out by the same analytical instrument 10.

According to embodiments disclosed herein, the test orders can be retrieved by the control unit 20 from an internal storage (such as a database), from a laboratory information system (LIS) and/or from a user interface communicatively connected to the control unit 20 or from a hospital information system (HIS) 50.

Each test order can comprise a processing priority indicative of a precedence by which the test order is to be carried out with respect to other test orders. As one example, processing priority may be defined on a two value scale such as “Urgent” and “Normal”, wherein increasing a priority from “Normal” results in “Urgent” and decreasing a priority from “Urgent” results in “Normal”, “Urgent” being considered in this case the highest level or processing priority. Alternatively, or additionally, the processing priority may be defined as a numerical scale, e.g., from 1 to 10, 10 being the highest priority level and 1 the lowest.

According to embodiments disclosed herein, when tests are ordered (usually by a medical practitioner), the processing priority is set either as ‘normal’ or ‘urgent/STAT’, STAT orders for analytical tests being issued for those tests that are needed immediately in order to manage medical emergencies.

In step 104, the control unit 20 can determine a processing workflow for processing the biological samples based on corresponding test order(s) and corresponding processing priority(s). The processing workflow can define a list such as, for example, a sequence of processing steps to be carried out by the plurality of laboratory instruments 10 in order to complete the test orders.

According to embodiments of the disclosed method/system, the processing workflow can also define one or more of the following:
Sequence in which the test orders are to be processed—The test order processing sequence can define the order in which the biological samples can be processed by the test orders.
Timing of processing of the test orders—According to embodiments of the disclosed method/system, in addition to the processing sequence, the timing according to which the biological samples or aliquots thereof are processed can also be defined by the processing workflow.

All-in-all, the processing workflow for processing each biological sample can be determined such that test order(s) with higher processing priority are scheduled to be completed before test order(s) with lower processing priority. It can be emphasized that due to unforeseeable events, such as an instrument malfunction, the actual processing may deviate from the processing workflow in terms of time of availability of the results and/or in the sequence of the processing steps.

In a step 106, the availability of analytical result(s) corresponding to each test order can be determined. The availability of analytical result(s) in this context can refer to the point in time when the control unit 20 can predict/forecast that the at least one analytical step on the biological sample is completed and the corresponding result becomes available. According to various analytical tests, an analytical result may not be immediately available after completion of the analytical step(s) on the biological sample, but only after a so-called validation of the measurement. In such cases, the forecast availability of analytical result(s) can take into account the time required for such validation.

According to embodiments disclosed herein, the step 106 of determining a forecast availability of an analytical result can comprise one or more substeps from the list comprising:
Determining a predicted instrument processing time—This substep refers to predicting the total time required by the laboratory instrument(s) 10 to process the biological sample to complete the corresponding test order. According to embodiments disclosed herein, the algorithm predicting the instrument processing time can take into account expected staff workload, throughput of instruments, downtimes due to scheduled and/or predicted maintenance activities. For example, historical data indicative of past processing time can be taken in consideration for predicting instrument processing time.
Determining a predicted validation time—The validation time can be determined in view of a schedule of a laboratory staff responsible for validation of analytical test results. In other words, the validation time can be correlated with the work schedule of the staff that performs the validation—at the time when the processing of the biological sample by the laboratory instrument(s) 10 is completed. In the current context, the term ‘schedule of a laboratory staff’ can encompass data representing work shifts of laboratory staff, periods of increased workload/demand. According to embodiments disclosed herein, the algorithm predicting the validation time can take into account time of day, day of week, holidays, periods with increased occurrence of epidemics, and the like. A degree of automation of result validation can be taken in consideration for predicting validation time.
Determining a predicted sample transportation time—This substep can comprise prediction of the period of time required for the biological sample(s) to be transported from a point of collection from the patient (e.g., by a phlebotomist) to the analytical laboratory 1. According to embodiments disclosed herein, the algorithm predicting the sample transportation time can take into account time of day, day of week, holidays, periods with increased occurrence of epidemics with higher than usual demand, traffic rush hours, and the like.
Determining delays due interdependency(s) between analytical step(s) and analytical results—Certain analytical steps can be performed based on/in reaction to results from other analytical steps, for example, reflex testing, reruns, confirmatory testing. These interdependencies can be taken in consideration in predicting forecast availability of an analytical result.

In a step 108, staff workload is determined at the time of the forecast availability(s) of the respective analytical test result(s). According to various embodiments, staff can comprise a physician responsible for diagnosing/treating the patient whose biological sample the respective analytical test result(s) correspond to. Within step 108, three substeps can be identified:
I) Identifying the relevant staff for a particular analytical result—According to embodiments disclosed herein, the relevant staff for a particular analytical result can be retrieved by the control unit 20 from a Hospital Information System (HIS) 50 and/or part of a test order and/or determined by a schedule of the health care institution. For example, in an emergency ward, the responsible staff can comprise all physicians on duty at the time of the forecast availability(s). On the other hand, for a specialist physician's office, the responsible staff can comprise all physicians of a respective specialty or even a particular physician treating/diagnosing the respective patient. In very simple terms, in this substep, the control unit 20 can determine who is “waiting” for this result.
II) Predicting the staff workload of the relevant staff—In this substep, a staff workload of the relevant staff can be determined for the moment in future when the respective analytical result(s) can be forecast to become available. The algorithm determining the staff workload can look ahead instead of merely relying on a current staff workload. This can ensure that the prediction of the staff workload is still relevant when the samples have been processed, the measurements validated and the results available for the staff. This determination can be used to evaluate the actual processing priority of a test order. For example, if a medical practitioner still has a high number of patients to consult before the patient corresponding to a particular test order, then the respective analytical results can “wait”. On the other hand, if by the time an analytical result is forecast to be available, the medical practitioner has no other patients to be treated/diagnosed, then the test order can be prioritized as otherwise the medical practitioner may have to wait for the test results which can be a waste of time and resources and potentially a risk for the patient.
III) Determining a workload threshold—The workload threshold can be indicative of a staff workload limit under which the test order is to be prioritized to avoid a test result from being delayed and above which the test order is to be de-prioritized to allow for more urgent test orders to be completed first. A workload threshold above 0 can act as a buffer to allow for some imprecision of the forecasting of result availability and still avoiding delayed respectively too early test results. A workload threshold of 0 can lead to just in time availability, but with no tolerance of the predictions. Another way to describe the workload threshold is to say that the medical practitioner may have the analytical results available a certain amount of time (a certain number of patients still to be treated) before he becomes free.

According to embodiments disclosed herein, the staff workload and the workload threshold can be indicative of one or more from the list comprising:
A number of “other” test results a medical practitioner has to review before reviewing the test result corresponding to the respective test order—In other words, the future backlog of medical practitioner can be determined. The term “other” in this context can refer to any test result different from the one for which the staff workload is currently being determined.
A predicted period of time from the forecast availability of analytical result(s) until a medical practitioner is to review the analytical result(s)—The period of time before a medical practitioner can be predicted to review the analytical result(s) can be determined based on a number of factors such as, but not limited to: a work schedule of the medical practitioner, work shifts, foreseen absences and/or more urgent planned activities. Since patients in critical condition are always diagnosed and/or treated first, the staff workload corresponding to a test order for the biological sample of a patient in critical condition is always 0 or ‘empty’, wherein a staff workload of 0 can ensure that the processing priority of corresponding test orders can be increased to the highest level of processing priority.
The severity of the patients of the medical practitioner—In addition to the number of the test results a medical practitioner has to review; the outcome of these respective test results can also be taken in consideration in calculating the staff workload of a medical practitioner. For example, if a medical practitioner has a high number of “bad” test results indicating very serious patient medical conditions, then his/her staff workload can be considered to be higher versus a doctor with his/her patient(s) showing “good” test results (i.e., no serious medical conditions) since treating a serious condition can take more time. The distinction between severe and less severe conditions can be determined according to know ranges for analytical tests results which aid to objectively determine whether the doctor has a serious case to solve (i.e., that takes more time).

According to further embodiments, the step of determining the staff workload can comprise the step of determining a “workload overflow” from a medical practitioner ending a work shift to another medical practitioner starting his/her work shift. The workload overflow can be added to the staff workload of the medical practitioner starting the work shift in that the test order can be assigned (even if temporarily) to more than one medical practitioner. Hence, the workload is not necessarily empty at the start of a work shift.

In a next step 110, the control unit 20 can compare the staff workload and the workload threshold:
If—at the time of forecast availability of the analytical result(s)—the staff workload is higher than the workload threshold, then the processing priority corresponding to the respective test order can be decreased in step 112. This case can correspond to a medical practitioner (e.g., doctor) with a backlog who may already have patients to diagnose/treat before needing this analytical result. Hence, the test order can be de-prioritized to allow for more urgent test orders to be processed by the analytical laboratory. It can be noted that a processing priority which is already at the lowest level in the defined range can, in this case, be maintained at this lowest level.
On the other hand, if—at the time of forecast availability of the analytical result(s)—the staff workload is lower than the workload threshold, then the processing priority corresponding to the respective test order can be increased in step 114. This case can correspond to a use case when the medical practitioner can be predicted to need the analytical results immediately or very shortly after they are forecast to become available. For example, if the staff workload threshold is defined as two patients and at the time of forecast availability, the medical practitioner only has one other patient waiting to be treated/diagnosed, that can mean that the analytical result may be needed in very short time after becoming available, actually in less time than the defined threshold (buffer). Therefore, the processing can be increased.
Once the staff workload matches the staff workload threshold, in step 116 the control unit 20 can controls the plurality of laboratory instruments 10 of the analytical laboratory 1 to process each biological sample according to the processing workflow. The term “match” can be interpreted, in the context of a staff workload matching the staff workload threshold, as being substantially equal to.

### EXAMPLE 1

for a medical practitioner treating an average of 4 patients per hour, an average forecasting deviation of 30 minutes, then a threshold of 2 patients would be determined. Hence, if the workload of the medical practitioner is 5 patients, it can mean the results may be sitting idle, therefore the test order may be deprioritized. On the other hand, if the workload of the medical practitioner is 1 patient, it can mean the results may have a risk to be available too late, therefore the test order can be prioritized.

### EXAMPLE 2

When a medical practitioner starts his/her work shift at 8 AM, the workload can be 0 or “empty” and the forecast availability of the analytical results can be 8:30 AM, this can mean the test order may be prioritized. However, if the forecast availability of the analytical results is 1:30 AM, then the analytical result may be early as the medical practitioner may be out of office and the test order can be deprioritized. Note: the above examples assume patients in non-critical condition.
The test results obtained by the at least one analytical step on the biological samples performed by the at least one of the plurality of the laboratory instruments 10 can then be reported step 118. According to various embodiments disclosed herein, the results can be reported by a data transfer to a hospital information system (HIS) 50, via a user interface of the control unit 20, by a data transfer to a cloud server accessible by a mobile device application of a medical practitioner, by way of a printout, or other suitable methods.

According to further embodiments disclosed herein, the step 116 of controlling the plurality of laboratory instruments 10 of the analytical laboratory 1 to process each biological sample according to the corresponding processing workflow can comprise the step of controlling an instrument for loading the biological samples such as to load the biological samples in the order of processing priority(s) thereof in order to avoid samples with lower processing priority to overload the laboratory instruments 10 and samples with higher priority being stuck.

In an embodiment of the analytical laboratory 1 wherein sample tubes comprising the biological samples are loaded into an instrument for loading samples in bulk, the instrument for loading samples can be configured to sort the sample tubes based on the processing priority of the corresponding test orders.

In a further embodiment of the analytical laboratory 1 wherein sample tubes comprising the biological samples are loaded into an instrument for loading samples in sample racks, the instrument for loading samples can be configured to individually load (pick) the sample tubes from the sample racks based on the processing priority of the corresponding test orders.

As illustrated in the flowcharts of FIGS. 1-7, according to embodiments disclosed herein, the processing workflow for processing each biological sample as well as the forecast availability of an analytical result corresponding to each test order can be re-determined each time the processing priority of one or more test order(s) is changed, i.e., increased or decreased. In other words, the method can be performed in iterations until an optimal solution can be determined. In this context, iteration can refer to the series of steps: 104 through 110. According to embodiments disclosed herein, a maximum number of iterations can be defined after which the plurality of laboratory instruments 10 of the analytical laboratory 1 are controlled by the control unit 20 to process each biological sample according to the corresponding processing workflow even if the staff workload does not match precisely the staff workload threshold.

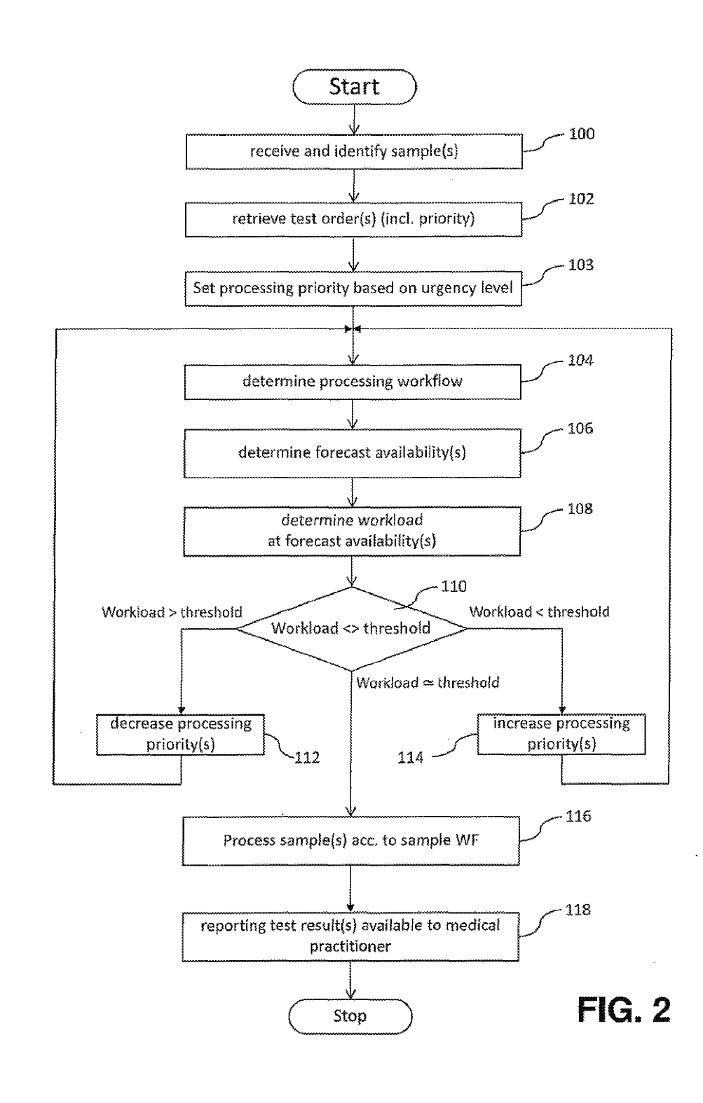

FIG. 2 shows a flowchart illustrating an embodiment of the disclosed method wherein the processing priority of a test order can be set—in step 103—based on a determined urgency level. The urgency level can be determined by the control unit 20 based on one or more from the list comprising:
Processing priority level of test order as retrieved—In order to ensure timely availability of results of analytical test(s), medical practitioners can order analytical test(s) on a sample of a patient as urgent whenever they decide that the particular analytical test or patient sample should be prioritized. This choice of priority by the medical practitioner can be weighted in when determining the actual urgency level.
An indication of patient criticality—Test orders related to patients in critical condition (e.g., heart attack, sepsis, stroke) are always prioritized and the urgency level can be determined accordingly. The indication of patient criticality may be either part of the test order or retrieved separately by the control unit 20 such as, for example, from a Hospital Information System (HIS) 50. Patient demographics can also be a factor influencing test order urgency.
Analytical test class—Certain types or classes of analytical tests are known to be ordered in life-threatening conditions. Therefore, the control unit 20 can determine the urgency level and set the processing priority accordingly—irrespective of staff workload. In addition, certain dependencies between various analytical tests can also influence test order urgency.
Patient condition progress/change—If the condition of a patient deteriorates, the control unit 20 can re-determine the urgency level and set the processing priority at its highest accordingly. Patient condition progress/change may be determined in particular using active patient monitoring devices communicatively connected to a Hospital Information System (HIS) 50, from where it can be retrieved by the control unit 20. For example, active patient monitoring can comprise (but not limited to): Connected ECG devices, respiratory function monitoring devices, blood oxygen level monitoring, pulse oximetry, arterial blood pressure monitoring, central venous pressure monitoring, continuous central venous oxygenation saturation monitoring, temperature monitoring. The patient monitoring can be either in a hospital setting or outside (including but not limited to patient home monitoring and patient monitoring in an ambulance). An advantage of considering patient condition using active patient monitoring can be that once the patient's condition significantly deteriorates, test orders for that patient can automatically be prioritized without the treating doctor having to contact the analytical laboratory.
Source of test order—There are certain use cases when the origin/source of a test order can play an important role in determining the urgency and therefore necessary processing priority of a test orders.
A status of one or more of the plurality of laboratory instruments 10—Changes in the operational status of laboratory instruments 10 can directly affect the availability of analytical results. Hence, these can be considered in setting the urgency and, hence, processing priority of test order. For example, if one of two instruments 10 of the same kind malfunctions, then some test orders can be de-prioritized to ensure the most urgent test are completed in time—despite the malfunctioning instrument 10.

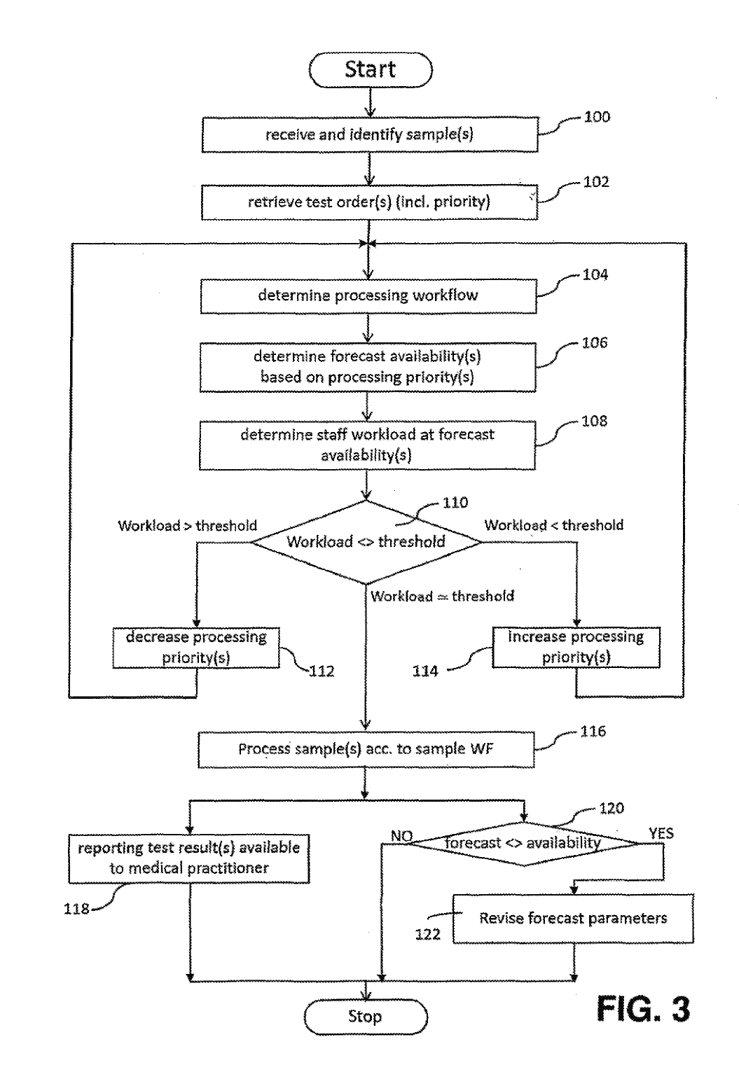

FIG. 3 shows a flowchart illustrating an embodiment of the disclosed method for operating an analytical laboratory 1, wherein machine learning can be employed to revise parameters for determining forecast availability of analytical results if the actual availability of analytical result(s) deviate from the forecast availability.

According to such further embodiments of the disclosed method/system, forecasting of the availability of analytical results corresponding to test orders can be based on historical data and a plurality of weighted parameters. Accordingly, the method can further comprise the steps:
Recording actual availability of analytical result(s)—In order to be able to evaluate the accuracy of the algorithm forecasting availability of analytical results, the actual time such are available can be recorded as a bases of comparison.
Recording data influencing the actual availability of analytical result(s)—There can be a number of factors that may influence the availability of the analytical results, comprising:
   Actual processing time of the biological samples by at least one of the plurality of the laboratory instruments 10;
   Actual validation time of a measurement—Actual period of time required for the biological sample(s) to be transported from a point of collection from the patient to the analytical laboratory 1.
If the actual availability of analytical result(s) deviates from the forecast availability of the same analytical results (step 120), then a correlation between the deviation of the actual availability of analytical result(s) from the forecast availability of the same analytical results and the influencing data can be determined. For example, if an analytical result actually becomes available 30 minutes later than forecast and at the same time the actual validation of the measurement took 30 minutes longer than predicted, then a correlation can be determined.
If a correlation is found, revising (step 122) one or more of the plurality of parameters used in the step of determining the forecast availability of analytical results. Continuing on the example of the preceding bullet point, since a correlation is found between delayed availability of the analytical results and the validation time, the parameter used in forecasting the validation time (e.g., average/mean) can be adjusted in view of the experienced delay. According to various embodiments, extreme values can be disregarded from consideration when revising parameters in order to avoid a single isolated occurrence from disrupting future calculations.

Actual can refer to real recorded values as opposed to predicted, forecast values. As such, actual values can be recorded and not calculated.

According to further embodiments, in addition to determining a correlation between the deviation of the actual availability of analytical result(s) from the forecast availability of the same analytical results and the influencing data, a determination of causality root-cause analysis can also be performed. Continuing further on the example of the paragraph above, if it is found that the laboratory instruments 10 finished processing the biological sample at e.g., 16:59 and knowing that laboratory personnel changes work shifts at 17:00, the cause for the delayed validation can be determined to lie with the timing of the validation with respect to work shifts. As a remedy, parameters of the forecasting algorithm can be adjusted to account for delays if a measurement needs to be validated in a time coinciding with a change of work shifts of laboratory personnel.

According to further embodiments, machine learning can be extended to optimizing the determination of the staff workload respectively the workload threshold by analyzing the activities of the medical practitioners. As part thereof, a time the medical practitioner takes to consult a patient can be recorded. Once a sufficient amount of records is available, a correlation can be determined between a deviation of the actual time the medical practitioner takes to consult a patient versus the forecast time that was considered in determining the staff workload. Based on this correlation, parameters used in determining the staff workload as well as the workload threshold can be adjusted iteratively to minimize the deviation. According to further embodiments, the prediction of the period of time from the forecast availability of analytical result(s) until a medical practitioner is to review the analytical result(s) can be specific to a medical practitioner and can be based on recorded work habits such as average/mean time to treat a patient. Additionally, the criticality of the patients can be considered (based on the analytical results) to distinguish between an average/mean time to treat a critical patient from the time to treat a mildly ill patient.

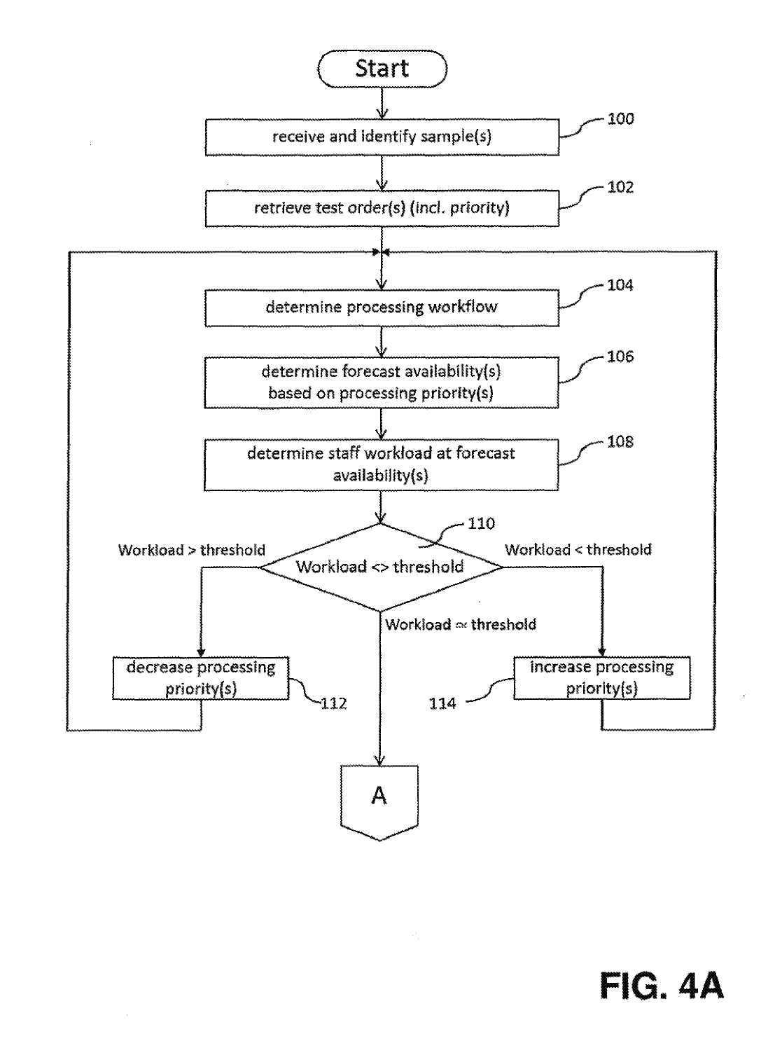

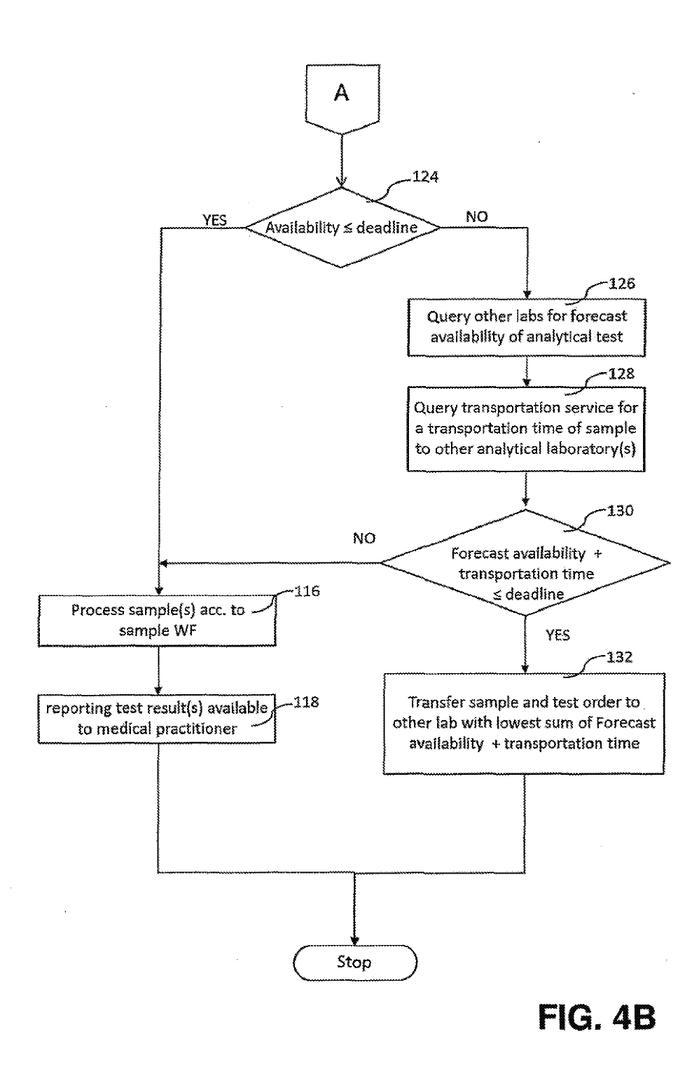

FIGS. 4A and 4B show a flowchart illustrating an embodiment of the disclosed method for operating an analytical laboratory, wherein the analytical laboratory 1 can be in communicative connection with one or more other analytical laboratory(s). As illustrated on the flowchart, if the forecast availability of analytical result(s) corresponding to the test order(s) for a biological sample is beyond a specified availability deadline (specified in particular in the test order by the medical practitioner who issued the test order), then other analytical laboratories can be contacted to evaluate if the test order can be completed in time by these. Hence, in step 124, the control unit 20 can determine whether the analytical laboratory 1 that received the sample is not able to complete a test order by a set deadline. If so, in step 126, the control unit 20 can send a query to the other analytical laboratory(s) for providing a forecast of the availability of the analytical results corresponding to the test order(s) if the biological sample can be processed by the further analytical laboratory(s). Then, in step 128, a query can be sent to one or more transportations services for a predicted transportation time of a sample to other analytical laboratory(s). Thereafter, in step 130, the control unit 20 can determine the sum of the forecast availability and predicted transportation time for each of the other analytical laboratory(s) and, in step 132, can transfer the biological sample to the other analytical laboratory for which the sum of the forecast availability and predicted transportation time are the lowest if lower than the specified availability deadline.

According to various embodiments, the biological sample can be transferred to a further analytical laboratory by a transportation service either manually (e.g., courier or taxi) and/or automatically by a sample transportation system (e.g., a pneumatic tube or aerial transport system, such as a drone).

According to various embodiments, biological samples to be transferred to a particular further analytical laboratory can be automatically transported by a sample transport system—such a conveyor-belt system—to a pick up station. According to an even further embodiment, the biological samples to be transferred to a particular further analytical laboratory can also be packaged automatically—grouped by destination laboratory such as, for example, onto a sample rack comprising an RFID tag, wherein the control unit 20 can record the corresponding test orders and processing priority onto the RFID tag.

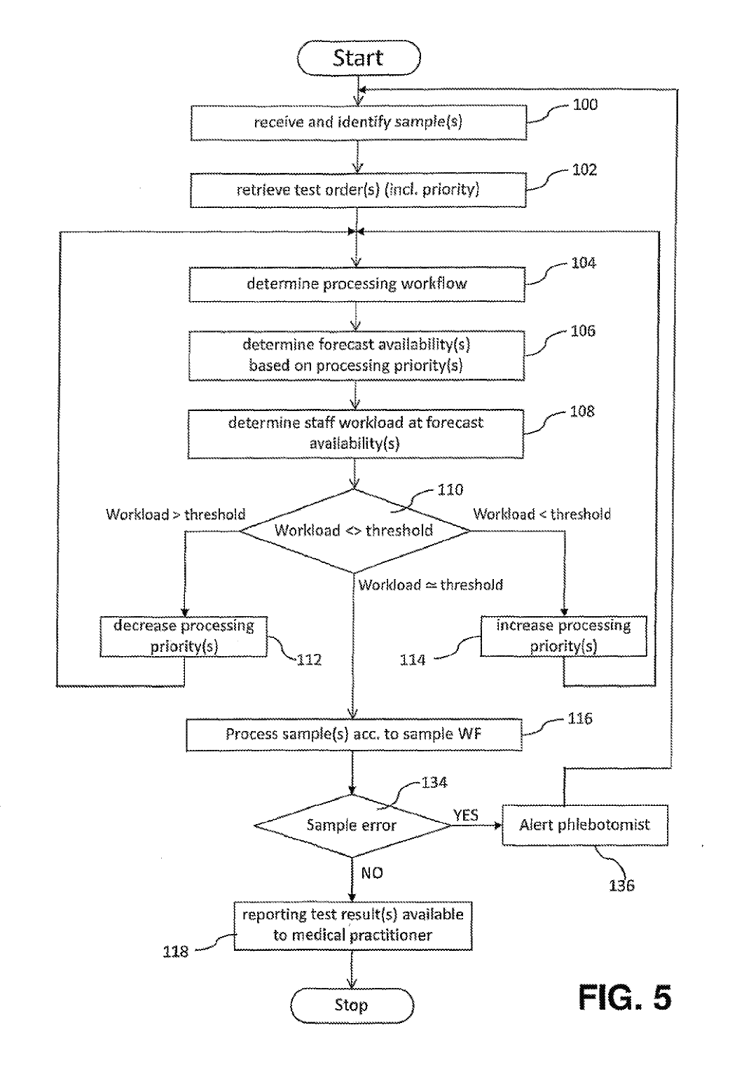

FIG. 5 shows a flowchart of an embodiment of the disclosed method addressing situations when at least one analytical step of the test order(s) cannot be performed on a biological sample due to sample quality/quantity. For saving time and resources, according to such embodiments, in step 134, the control unit 20 can detect if an analytical step cannot be performed on a biological sample due to sample quality/quantity. When this is the case, an alert can be raised, in step 136, indicating that a new sample needs to be provided for the analytical laboratory 1 to be able to carry out the test order(s). Thereafter, the processing workflow for this sample can be recalculated once the new sample is received (step 100). According to further embodiments, the processing priority can be increased for a test order as soon as a sample error is detected—in view of delays caused by the need to collect a new patient sample.

According to further embodiments, an alert can also be sent (e.g., to a phlebotomist) if a test order is indicative of a repetitive analytical test (e.g., Troponin test for cardiac care) requiring repeated sample collection from a patient based on a schedule. To assist medical practitioners in collecting the patient samples in a timely manner, the alert can be sent based on the schedule of the repetitive analytical test.

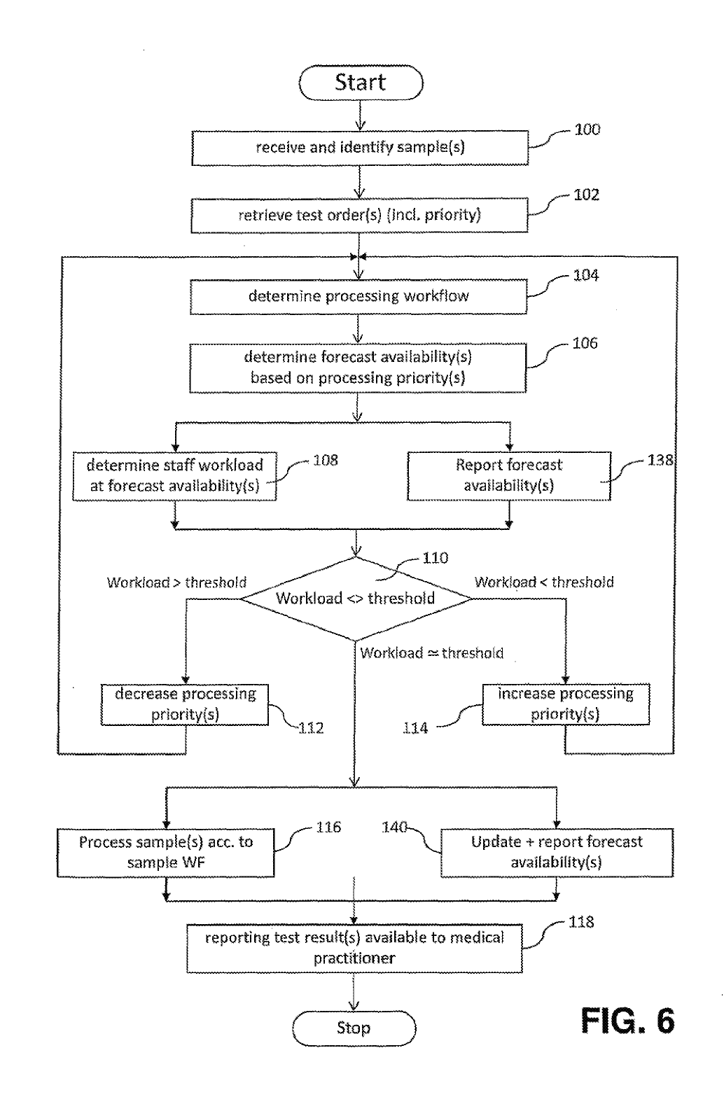

FIG. 6 shows a flowchart illustrating an embodiment of the disclosed method for operating an analytical laboratory 1, wherein the forecast availability(s) of analytical result(s) can be reported. In order to facilitate the planning activities of medical practitioners, as soon as the availability of an analytical result is determined, the forecast can be reported (step 138). To ensure the medical practitioner has the most up-to-date information, in a step 140, the forecast availability can be updated during the processing of the biological sample and the updated forecast can be reported.

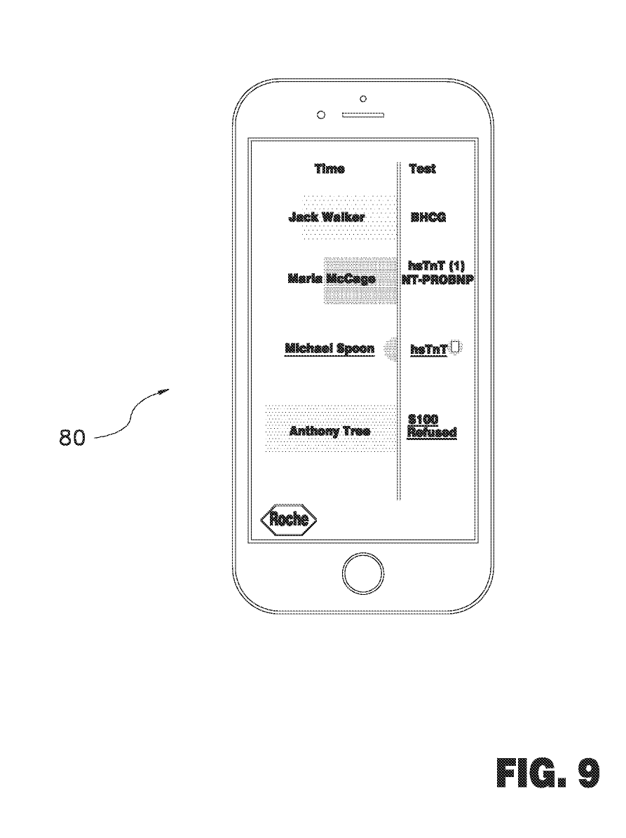

According to various embodiments of the disclosed method/system reporting of forecast availability of analytical result(s) can be via a user interface of a Hospital Information System (HIS) 50, by a data transfer to a cloud server accessible by a mobile device application of a medical practitioner, by way of a printout or other suitable methods. FIG. 9 shows a mockup of an example of a mobile device application reporting the forecast availability of analytical results in the form of progress bars, which can be updated (continuously or at certain intervals).

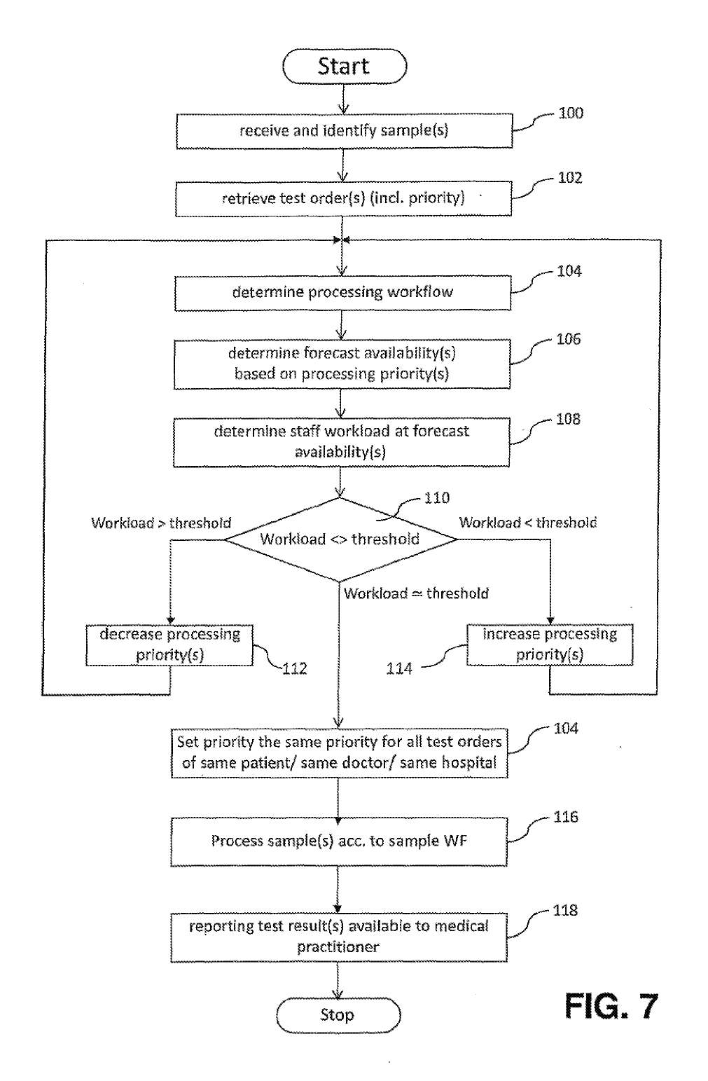

FIG. 7 depicts a flowchart illustrating an embodiment of the disclosed method for operating an analytical laboratory 1, wherein the processing priority level of all test orders in a group can be set to the highest level amongst the test orders of the group. Such grouping of test orders can be based on one or more from the list comprising:
Same patient—In order to support medical practitioners making a diagnosis and treating a patient in view of the “full picture”—that is in view of all analytical results related to that patient, all test orders related to the same patient can be prioritized equally, namely on the highest priority level amongst the test orders for the patient.
Same medical practitioner who is to review the analytical results—There are certain situations when there is a need for all test orders for a particular medical practitioner who is to review a number of analytical results to be available in the same time frame. The reasons can include: medical practitioners who are known to always handle emergency cases, medical practitioners known to be present to review analytical results only for a limited amount of time.
Same institution—In order to support scientific studies, where a certain number of analytical test results are evaluated, embodiments of the disclosed method can comprise the step of assigning the same processing priority for all test orders of the institution to ensure that all analytical results for the study are available in the same time frame and not one-by-one.

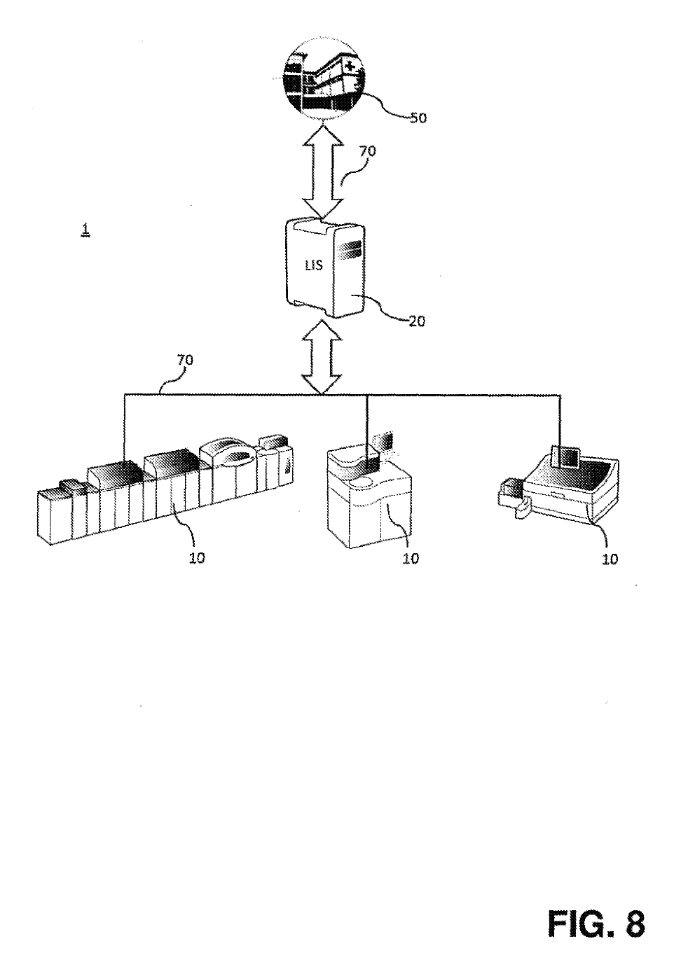

Turning now onto the block diagram of FIG. 8, embodiments of the disclosed analytical laboratory 1 for processing biological sample(s) can comprise a plurality of laboratory instruments 10 and a control unit 20 communicatively connected by a communication network 70. The plurality of laboratory instruments 10 can be configured to execute processing steps on the biological samples according to instructions from the control unit 20. According to embodiments of the disclosed analytical laboratory 1, the plurality of laboratory instruments 10 may be one or more instruments from the list comprising pre-analytical instruments, laboratory instrument(s) for loading samples comprising a sample input station, post-analytical instruments and also analytical instruments.

According to various embodiments of the disclosed analytical laboratory 1, the plurality of laboratory instruments 10 may be identical or different instruments such as clinical- & immunochemistry analyzers, coagulation chemistry analyzers, immunochemistry analyzers, urine analyzers, nucleic acid analyzers, hematology instruments and the like.

According to further embodiments disclosed herein, in view of data privacy regulations, patient and/or medical practitioner's personal information can be anonymized if required, ensuring that no patient and/or medical practitioner identity can be traced back.

Further disclosed and proposed is a computer program including computer-executable instructions for performing the method enclosed herein when the program can be executed on a computer or computer network. Specifically, the computer program may be stored on a computer-readable data carrier. Thus, specifically, one, more than one or even all of method steps as indicated above may be performed by using a computer or a computer network, preferably by using a computer program.

Further disclosed and proposed is a computer program product having program code, in order to perform the method disclosed herein in one or more of the embodiments enclosed herein when the program is executed on a computer or computer network. Specifically, the program code may be stored on a computer-readable data carrier.

Further disclosed and proposed is a data carrier having a data structure stored thereon, which, after loading into a computer or computer network, such as into a working memory or main memory of the computer or computer network, may execute the method according to one or more of the embodiments disclosed herein.

Further disclosed and proposed is a computer program product with program code stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Further disclosed and proposed is a modulated data signal which contains instructions readable by a computer system or computer network, for performing the method according to one or more of the embodiments disclosed herein.

Referring to the computer-implemented aspects, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Furthermore, hereby disclosed and proposed are:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising a program for performing the method according to one of the embodiments described in this description while the computer program is being executed on a computer or on a computer network,
a computer program comprising a program according to the preceding embodiment, wherein the program is stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code, wherein the program code can be stored or are stored on a storage medium, for performing the method according to one of the embodiments described in this description, if the program code is executed on a computer or on a computer network.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A computer implemented method for operating an analytical laboratory, the analytical laboratory comprising a plurality of laboratory instruments configured to obtain a measurement value indicative of a characteristic of the biological sample, the method comprising:
receiving and identifying a plurality of biological samples;
retrieving one or more test orders corresponding to each biological sample, each test order defining at least one analytical step to be carried out on the biological sample by at least one of the plurality of the laboratory instruments, each test order comprising a processing priority;
determining a processing workflow for processing each biological sample based on corresponding test order(s) and corresponding processing priority, wherein the processing workflow is determined such that test order(s) with higher processing priority are completed before test order(s) with lower processing priority;
determining a forecast availability of analytical result(s) corresponding to each test order;
determining a staff workload and a workload threshold at the time of forecast availability corresponding to each test order;
decreasing the processing priority corresponding to the respective test order if the staff workload is greater than a workload threshold;
increasing the processing priority corresponding to the respective test order if the staff workload is lower than the workload threshold; and
controlling the plurality of laboratory instruments of the analytical laboratory to process each biological sample according to the corresponding processing workflow and reporting test results obtained by the at least one analytical step on the biological samples performed by the at least one of the plurality of the laboratory instruments if the staff workload matches the workload threshold.

2. The method according to claim 1, wherein the staff workload and workload threshold are indicative of one or more from the list comprising:
a number of results a medical practitioner is to review before reviewing the test result corresponding to the respective test order, and/or
a predicted period of time from the forecast availability of analytical result(s) until a medical practitioner is to review the analytical result(s).

3. The method according to claim 2, wherein the staff workload corresponding to a test order for the biological sample of a patient in critical condition being always 0 and wherein a staff workload of 0 ensures that the processing priority of corresponding test orders are increased to a highest level of processing priority.

4. The method according to claim 1, further comprising,
setting the priority of a test order in view of an urgency level determined based on one or more from the list comprising:
processing priority level of the test order as retrieved,
an indication of patient criticality,
analytical test class,
patient condition progress/change, in particular based on patient monitoring, source of test order, and/or
a status of one or more of the plurality of laboratory instruments.

5. The method according to claim 1, further comprising,
re-determining a processing workflow for processing each biological sample; and
re-determining the forecast availability of an analytical result corresponding to each test order each time the processing priority of one or more test order(s) is increased or decreased.

6. The method according to claim 1, wherein determining a forecast availability of an analytical result comprises one or more steps from the list comprising:
determining a predicted processing time of the biological samples by the laboratory instrument(s),
determining a predicted validation time, wherein the validation time is determined in view of a schedule of a laboratory staff responsible for validation of analytical test results,
determining a predicted period of time required for the biological sample(s) to be transported from a point of collection from the patient to the analytical laboratory; and/or
determining delays due interdependency(s) between analytical step(s) and analytical results.

7. The method according to claim 1, further comprising:
reporting the forecast availability(s) of analytical result(s) upon being determined and/or re-determined.

8. The method according to claim 1, further comprising,
setting the processing priority level of all test orders to the highest level amongst the test orders corresponding to: same patient, same medical practitioner who is to review the analytical results corresponding to each test order, and/or same institution as originator of the test orders.

9. The method according to claim 1, wherein if forecasting of the availability of an analytical result corresponding to each test order is based on historical data and a plurality of weighted parameters, the method, further comprising,
recording actual availability of analytical result(s);
recording data influencing the actual availability of analytical result(s), the data comprising actual processing time of the biological samples by at least one of the plurality of the laboratory instruments, actual validation time, and actual period of time required for the biological sample(s) to be transported from a point of collection from the patient to the analytical laboratory;
determining a correlation between a deviation of the actual availability of analytical result(s) from the forecast availability of the same analytical results and the influencing data; and
revising one or more of the plurality of parameters used in the step of determining the forecast availability of analytical results if the actual availability of analytical result(s) deviate from the forecast availability of the same analytical results based on said correlation.

10. The method according to claim 9, further comprising,
recording time the medical practitioner takes to consult a patient; and
determining a deviation of the actual time the medical practitioner takes to consult a patient versus the forecast time that was considered in determining the staff workload.

11. The method according to claim 1, wherein if the forecast availability of analytical result(s) corresponding to the test order(s) for a biological sample is beyond a specified availability deadline, the method further comprises,
query one or more other analytical laboratory(s) for a forecast availability by the further analytical laboratory(s) of analytical results corresponding to the test order(s);
query a transportation service for a predicted transportation time of the biological sample to said other analytical laboratory(s); and
transferring the biological sample(s) to the other analytical laboratory for which the sum of the forecast availability and predicted transportation time is the lowest if lower than the specified availability deadline.

12. The method according to claim 1, further comprising,
generating an alert if at least one analytical step of the test order(s) cannot be performed on a biological sample due to its quality and/or quantity, the alert being indicative that a new sample need to be provided for the analytical laboratory to be able to carry out the test order(s) related to the biological sample.

13. An analytical laboratory, the analytical laboratory comprising:
a plurality of laboratory instruments for processing biological samples; and
a control unit, wherein the plurality of laboratory instruments and the control unit are communicatively connected by a communication network, wherein the control unit is communicatively connected by a communication network to a Hospital Information System and wherein the control unit is configured to carry out the method according to claim 1.

14. A computer program product comprising instructions which, when executed by a computer system, control an analytical laboratory to perform the steps of the method according to claim 1.
